(19) United States
(12) Patent Application Publication
(10) Pub. No.: US 2023/0338660 A1
(43) Pub. Date: Oct. 26, 2023

(54) VIAL AND SYRINGE ALIGNMENT GUIDE

(71) Applicant: Patrick A. Tomsha, Chewelah, WA (US)

(72) Inventor: Patrick A. Tomsha, Chewelah, WA (US)

(21) Appl. No.: 18/139,483

(22) Filed: Apr. 26, 2023

Related U.S. Application Data
(60) Provisional application No. 63/334,770, filed on Apr. 26, 2022.

(51) Int. Cl. A61M 5/24

(52) U.S. Cl. CPC A61M 5/2422; A61M 2005/2433; A61M 2005/2403

(57) ABSTRACT

Described is a vial and syringe alignment guide that is designed to assist a user in filling a syringe from a vial. The guide includes both a vial attachment and syringe attachment. When a vial is affixed with the vial attachment, a syringe placed within the syringe attachment is aligned with the vial to allow the user to safely draw fluid from the vial.

### CROSS-REFERENCE TO RELATED APPLICATIONS

This is a non-provisional application of U.S. Provisional Application No. 63/334,770, filed Apr. 26, 2022, the entirety of which is incorporated herein by reference.

### BACKGROUND OF THE INVENTION

### (1) Field of Invention

The present invention relates to a vial and syringe holder and, more specifically, to an alignment guide designed to assist a user in filling a syringe from a vial.

### (2) Description of Related Art

Vials are often filled with various medications that need to be injected intravenously. In operation, a user pierces the tip of the vial with a syringe while drawing in fluid from the vial into the barrel of the syringe. Such a process is easily accomplished by those individuals with firm hands. However, those with unsteady hands or poor dexterity can find it extremely difficult and often dangerous to perform such a task. As can be appreciated by those skilled in the art, filling a syringe from a vial can be dangerous if proper procedures are not followed. Nevertheless, even when performing the proper procedures, the process is prone to human error and risk.

Thus, a continuing need exists for a device to assist users in filling syringes from a vial to eliminate the risks associated with such a procedure.

### SUMMARY OF INVENTION

The present invention relates to a vial and syringe alignment guide adapted to assist a user in filling a syringe from a vial. The guide includes a base having both a vial attachment and a syringe attachment. The vial attachment is adapted to secure a vial therewith while the syringe attachment is adapted to hold a syringe while aligning a needle of the syringe with the vial. For example, the vial attachment includes a pair of flexible clips formed to secure hold a vial therebetween, while the syringe attachment includes an elongated arcuate trough. A syringe adapter can be include that is adapted to fit within the syringe attachment. The syringe adapter has an elongated arcuate channel for receiving a syringe therein.

In another aspect, flexible snap attachments rise from the syringe attachment. The flexible snap attachments are adapted to securely affix the syringe adapter to the syringe attachment.

In another aspect, a vial adapter is included. The vial adapter is a cylindrical sleeve terminating in a backstop with vent holes.

Additionally, in another aspect, a storage compartment is detachably attachable to the base.

Finally, as can be appreciated by one in the art, the present invention also comprises a method for forming and using the invention as described herein. For example, the method includes acts of positioning a syringe within the syringe attachment, while a vial is affixed in the vial attachment. Thereafter, the method includes an act of sliding the syringe toward the vial to cause a needle of the syringe to pierce the vial, allowing the user to withdraw fluid from the vial by withdrawing the plunger on the syringe.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 is an elevated view of a vial and syringe alignment guide according to various aspects of the present invention, depicting both a vial and syringe as positioned upon the alignment guide;

FIG. 2 is a side-view illustration of the alignment guide, depicting alignment of a syringe with a vial;

FIG. 3 is a side-view illustration of the alignment guide, depicting an aspect that includes a storage container;

FIG. 4 is an elevated view the alignment guide according to various aspects of the present invention, depicting an aspect that includes a syringe adapter, a vial adapter, and a storage container;

FIG. 5 is a side-view illustration of the alignment guide;

FIG. 6 is a top-view illustration of the alignment guide; and

FIG. 7 is a front-view illustration of the alignment guide.

### DETAILED DESCRIPTION

The present invention relates to a vial and syringe holder and, more specifically, to an alignment guide designed to assist a user in filling a syringe from a vial. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments.

Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

### (1) Description

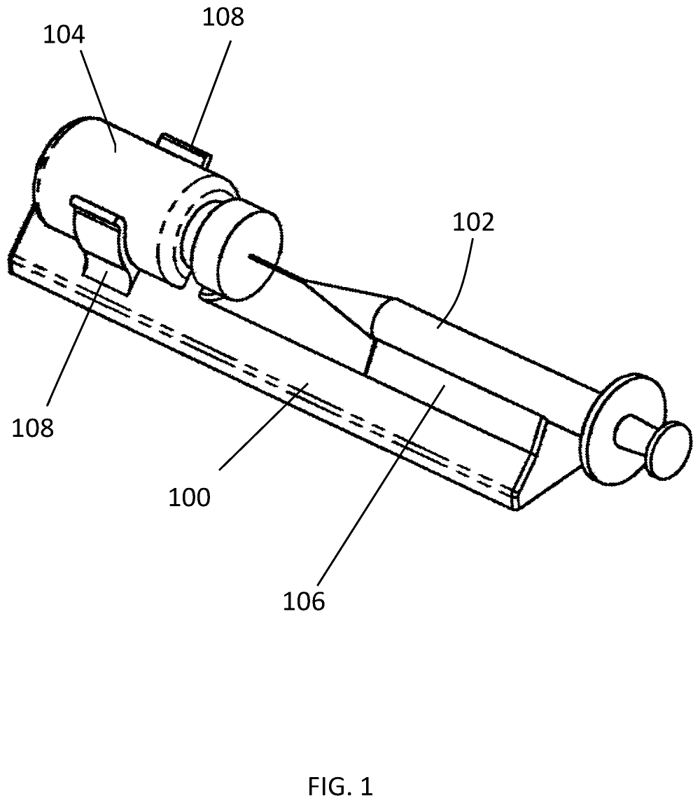

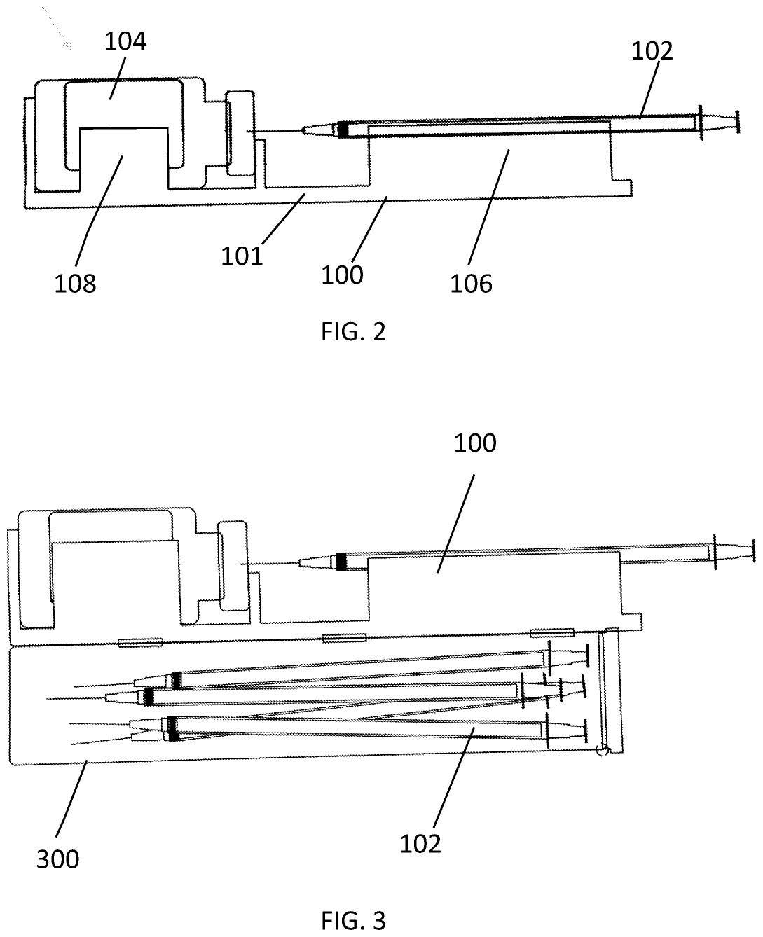

As shown in FIGS. 1 and 2, the present disclosure provides an alignment guide 100 that is used to align a syringe 102 with a medicine vial 104. The alignment guide 100 is designed to assist users in filling the syringe 102 from the vial 104 to eliminate the risks associated with such a procedure. Generally speaking, the alignment guide 100 has a base 101 with both a syringe attachment 106 and a vial attachment 108. Further details are provided below.

As can be appreciated by those skilled in the art, the vial attachment 108 is adapted to securely hold a vial 104. Thus, the vial attachment 108 is any mechanism or device that is operably for securely holding old the vial 104. As a non-limiting example and as depicted, the vial attachment 108 is pair of flexible vial clips that are formed to wrap partially around and secure the vial 104 therebetween.

As noted above, the alignment guide 100 also includes a syringe attachment 106 that is formed in any suitable manner to accommodate a syringe 102 and align the syringe 102 with the vial 104 to allow a user to easily and safely pierce the vial 104 and draw fluid from the vial 104. As a non-limiting example, the syringe attachment 106 has an elongated arcuate trough that allows the barrel of the syringe 102 to securely rest within the trough and align the needle of the syringe 102 with the vial 104.

The alignment guide 100 can also be devised to include several additional features. For example and as shown in FIG. 3, the alignment guide 100 can be formed to included a storage compartment 300 to allow for the storage of syringes 102 or other items (e.g., additional vials of medicine, etc.). In yet another aspect, the alignment guide 100 can include multiple storage compartments, such as one for new syringes and another for used syringes, etc.

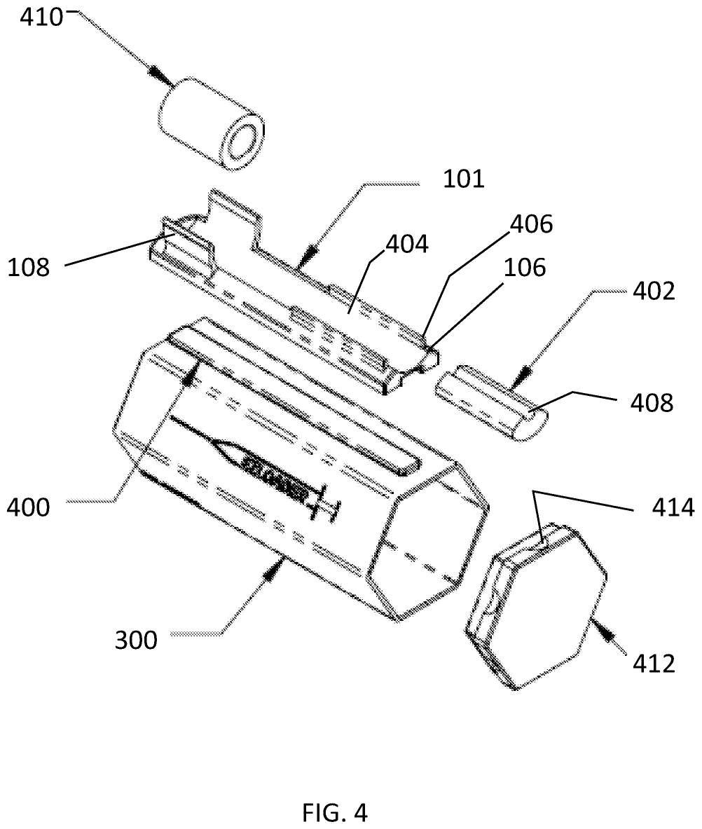

Yet another example is depicted in FIG. 4. As shown, the alignment guide base 101 is detachably attachable with a storage compartment 300 using any suitable mechanism or device. For example, a dovetail connector 400 can be formed in a portion of the storage compartment 300 to allow the base 101 to slide over and become affixed to the dovetail connector 400. Also shown is a syringe adapter 402. The syringe adapter 402 can be formed in various sizes to match common syringe sizing. Notably, the syringe adapter 402 is detachably attachable with the base 101 to allow the guide 100 to accommodate different syringe sizes. The syringe adapter 402 can be selectively attached/detached to the base 101 using any suitable mechanism or device. As a non-limiting example, rising from the sides of the arcuate trough 404 of the syringe attachment 106 are flexible snap attachments 406. The flexible snap attachments 406 are formed to allow the syringe adapter 402 to slide therebetween while having a snap or catch shape that prevents the syringe adapter 406 from popping or falling out of the syringe attachment 106. The syringe adapter 402 similarly has an elongated arcuate channel 408 that is adapted to allow a syringe to rest therein.

A vial adapter 410 can also be included that is designed to affix with the vial attachment 108. Notably, the vial adapter 410 is a sleeve or other item that can be affixed with a vial of various sizes to allow the vial to be secured in place against the vial attachment 108.

A cover 412 (e.g., lid or cap) is also included to enclose or otherwise seal the storage compartment 300. Grip points 414, such as finger indents, can be formed in the cover 412 to assist a user in easily attaching and removing the cover 412 from the storage compartment 300. Thus, using the storage compartment 300, a user can easily remove the cover 412 to store new or used syringes, vials, etc.

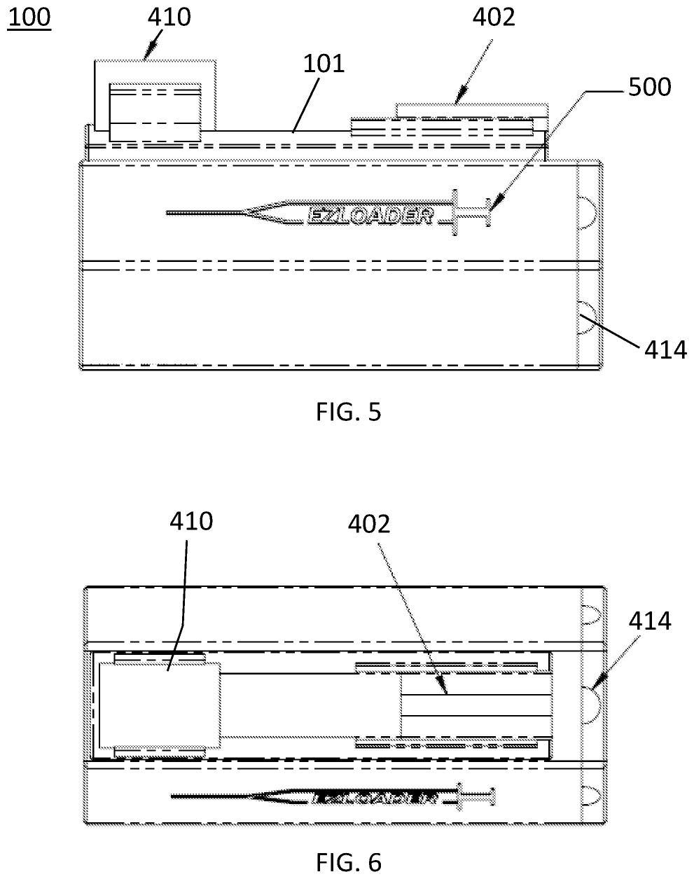

For further understanding, FIGS. 5 and 6 provide side and top-view illustrations, respectively, of the alignment guide 100. As shown, both the syringe adapter 402 and vial adapter 410 are securely affixed with the base 101. Also depicted is a logo 500 formed or otherwise affixed to a side of the storage compartment 300. The logo 500 provides art or branding as desired. The grip points 414 are also shown to allow ease of use by the user.

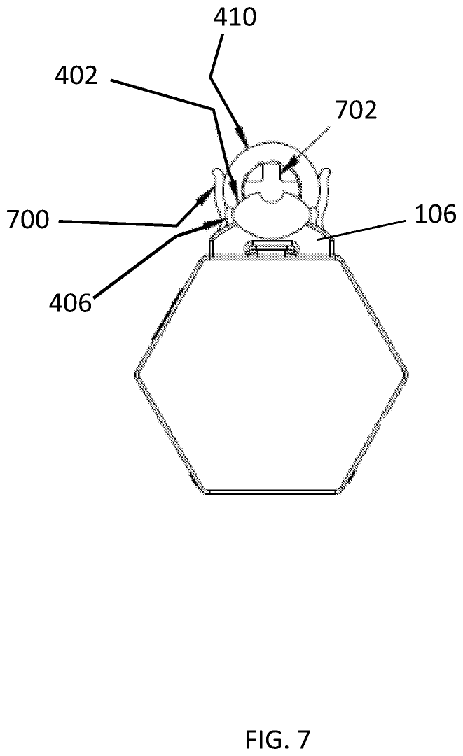

As noted above and as shown in FIG. 7, the syringe adapter 402 and vial adapter 410 are affixed in place using any suitable mechanism or device, such as flexible snap attachments 406 and flexible vial clips 700. In some aspects, the vial adapter 410 includes a backstop 702 with vent holes. Thus, when a vial is positioned within the vial adapter 410, the backstop 702 prevents the vial from moving away from the syringe when the syringe is pressed into the vial. The vent holes also allow for breathing when the vial is pressed within the vial adapter 410 to avoid air being trapped within the vial adapter 410, which could otherwise prevent a user from pressing the vial into the vial adapter 410. As can be appreciated by those skilled in the art, with the vial secured within the vial adapter 410, a user can then easily insert a syringe into the vial. For example, in some aspects, a user can easily pierce the vial with the needle of the syringe at an angle and then gently rest the syringe upon the trough of the syringe attachment 106 or syringe adapter 402. In other aspects, a user can place the syringe upon the syringe attachment 106 or syringe adapter 402 and then slide the syringe forward to cause the needle to pierce the vial.

Finally, while this invention has been described in terms of several embodiments, one of ordinary skill in the art will readily recognize that the invention may have other applications in other environments. It should be noted that many embodiments and implementations are possible. Further, the following claims are in no way intended to limit the scope of the present invention to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”. Further, while particular method steps have been recited in a particular order, the method steps may occur in any desired order and fall within the scope of the present invention.

What is claimed is:

1. A vial and syringe alignment guide, comprising:
a base, the base having both a vial attachment and a syringe attachment, the vial attachment being adapted to secure a vial therewith while the syringe attachment is adapted to hold a syringe while aligning a needle of the syringe with the vial.

2. The vial and syringe alignment guide as set forth in claim 1, wherein the vial attachment includes a pair of flexible clips formed to secure hold a vial therebetween.

3. The vial and syringe alignment guide as set forth in claim 2, wherein the syringe attachment includes an elongated arcuate trough.

4. The vial and syringe alignment guide as set forth in claim 3, further comprising a syringe adapter, the syringe adapter being adapted to fit within the syringe attachment, the syringe adapter having an elongated arcuate channel for receiving a syringe therein.

5. The vial and syringe alignment guide as set forth in claim 4, further comprising flexible snap attachments rising from the syringe attachment, the flexible snap attachments adapted to securely affix the syringe adapter to the syringe attachment.

6. The vial and syringe alignment guide as set forth in claim 5, further comprising a vial adapter, the vial adapter being a cylindrical sleeve terminating in a backstop with vent holes.

7. The vial and syringe alignment guide as set forth in claim 6, further comprising a storage compartment, the storage compartment being detachably attachable to the base.

8. The vial and syringe alignment guide as set forth in claim 1, further comprising a syringe adapter, the syringe adapter being adapted to fit within the syringe attachment, the syringe adapter having an elongated arcuate channel for receiving a syringe therein.

9. The vial and syringe alignment guide as set forth in claim 8, further comprising flexible snap attachments rising from the syringe attachment, the flexible snap attachments adapted to securely affix the syringe adapter to the syringe attachment.

10. The vial and syringe alignment guide as set forth in claim 1, wherein the vial attachment includes a pair of flexible clips formed to secure hold a vial therebetween.

11. The vial and syringe alignment guide as set forth in claim 1, wherein the syringe attachment includes an elongated arcuate trough.

12. The vial and syringe alignment guide as set forth in claim 1, further comprising a vial adapter, the vial adapter being a cylindrical sleeve terminating in a backstop with vent holes.

13. The vial and syringe alignment guide as set forth in claim 1, further comprising a storage compartment, the storage compartment being detachably attachable to the base.

14. A method for filling a syringe from a vial using a vial and syringe alignment guide, comprising acts of:
affixing a vial with a vial attachment on the vial and syringe alignment guide;
positioning a syringe within a syringe attachment on the vial and syringe alignment guide;
moving the syringe toward the vial to cause a needle of the syringe to pierce the vial; and
withdrawing a plunger on the syringe to draw fluid from the vial.
